(19) United States
(12) Patent Application Publication
(10) Pub. No.: US 2020/0268809 A1
(43) Pub. Date: Aug. 27, 2020

(54) Health Supplement Compositions

(71) Applicant: Ravi Ramamoorthy Iyer, Vienna, VA (US)

(72) Inventor: Ravi Ramamoorthy Iyer, Vienna, VA (US)

(21) Appl. No.: 16/788,565

(22) Filed: Feb. 12, 2020

Related U.S. Application Data
(60) Provisional application No. 62/811,296, filed on Feb. 27, 2019.

(51) Int. Cl. A61K 35/618; A61K 36/54; A61K 31/375; A61K 36/9066; A61K 36/67; A61K 47/46; A61K 9/00; A61K 36/324; A61K 36/185; A61K 35/747; A61K 47/02; A61K 35/616; A61K 31/728; A61K 31/195; A61K 36/31; A61K 36/40; A61K 36/8945; A61K 36/88; A61K 36/71

(52) U.S. Cl. CPC A61K 35/618; A61K 36/71; A61K 31/375; A61K 36/9066; A61K 36/67; A61K 47/46; A61K 9/0053; A61K 36/324; A61K 36/185; A61K 35/747; A61K 47/02; A61K 35/616; A61K 31/728; A61K 31/195; A61K 36/31; A61K 36/40; A61K 36/8945; A61K 36/88; A61K 36/54

(57) ABSTRACT

Various embodiments of health supplements for mammals are disclosed. The health supplement consists of one or more metabolite ingestion components; anti-inflammatory components; and oral absorption components. The metabolite ingestion component includes green-lipped mussel powder in an amount between 1-5 mg/kg of body weight. The anti-inflammatory component includes cinnamon powder in an amount between 0.1-1. mg/kg of body weight; ascorbic acid powder; turmeric powder present in an amount between 0.1-1.0 mg/kg of body weight; Piper nigrum powder in an amount between 0.05-0.2 mg/kg of body weight; and the oral absorption component includes diatomaceous earth powder and molasses powder. The metabolite ingestion component includes sea cucumber powder, sodium hyaluronate powder, methylsylfonylmethane powder, and creatine monohydrate powder. The anti-inflammatory component includes Boswellia serrata powder, Harpagophytum procumbens powder, and calcined curcumin powder. The oral absorption component includes Lactobacillus probiotic powder; and calcined dolomite powder.

### CROSS-REFERENCE TO RELATED APPLICATIONS

This application claims priority to U.S. Provisional Application No. 62/811,296 filed Feb. 27, 2019, which is hereby incorporated herein by reference.

### FIELD OF THE INVENTION

The present invention relates generally to health supplements. More specifically, the health supplement compositions are disclosed herein.

### BACKGROUND OF THE INVENTION

The complexity of pain results in a variety of treatment options—medication, therapies, and mind-body techniques. For example, in companion animals, muscle and joint problems are significant causes of diminished vitality. Pain management is a branch of medicine that applies science to the reduction of pain. It covers a wide spectrum of conditions including neuropathic pain, sciatica, postoperative pain and more. Turning now to fertility, which is a point of interest among human as well as animal breeders. In the post-urban era, couples increasingly desire to postpone reproductive and parental roles to later years, which place an increasing burden on the fertility of aging couples. Among breeders of companion animals, obtaining and/or maintaining reproductive health continues to be a focal point. Even more, the general population's advancing age brings with it, for example, issues with age-related cognitive decline as well as issues with maintaining cognitive function and an active lifestyle. However, musculoskeletal decline typically increases with advancing age. Unfortunately, current solutions known in the art for connective tissue, target tissue, and/or organ health typically address the issues from a unidimensional approach.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

Other technical advantages may become readily apparent to one of ordinary skill in the art after review of the following figures and description. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

The present disclosure includes many aspects and features. Moreover, while many aspects and features relate to, and are described in the context of detection of presence of one or more intruder devices, embodiments of the present disclosure are not limited to use only in this context.

The complexity of pain results in a variety of treatment options—medication, therapies, and mind-body techniques. For example, in companion animals, muscle and joint problems are a significant cause of diminished vitality. Pain management is a branch of medicine that applies science to the reduction of pain. It covers a wide spectrum of conditions including neuropathic pain, sciatica, postoperative pain and more.

Turning now to fertility, which is a point of interest among human as well as animal breeders. In the post urban era, couples increasingly desire to postpone reproductive and parental roles to later years and these choices place increasing burdens on the fertility of aging couples. Companion animal breeders typically seek to obtain and/or maintain reproductive health.

Finally, the advancing age of the general population worldwide brings with it, for example, the problem of age-related cognitive decline and the need to maintain cognitive function for the maximal duration in a lifetime. There has also been an increased focus on the maintenance of an active lifestyle, which may involve sports and weekend athletic activities, to maintain health and vitality. Not to be restricted by theory, the toxic effects of reactive free radicals generated as a byproduct of normal metabolism as well as injuries are believed to be a major cause of the process of aging and age-related functional decline. At the musculoskeletal level, micro-injuries to muscles, ligaments, and joints are recognized as the normal burden of activities of daily living. For example, microtrauma injuries result from frequent, repeated use of muscles, tendons, ligaments, joints, and bones. Microtrauma injuries include, but are not limited to, small tears in fibers and connective tissue of the muscles as well as sprained ligaments, strained muscles, and overstretched tendons.

The rate of these micro-injuries typically increase with both age and the activity level of the individual. The cumulative effect of this process is believed to be what constitutes aging at the tissue level. However, the process of aging, like all processes, can be manipulated and tweaked to maximize the functional lifespan. For example, apart from the genetics of the individual, some modifiable factors are believed to be critical in slowing down the aging process, including, but not limited to, blood flow to target tissues, active inflammation, and nutrient bioavailability. For example, tissues that receive increased blood flow typically exhibit decreased healing times, a reduction in degenerative functionality, and an increase in vitality. In addition, tissue that exhibits reduced inflammation and/or damage from reactive free radicals are typically best positioned to benefit from the administration of any nutritional supplements. The principles of blood supply restriction, toxic radical injury and micro-inflammation are recognized as components of the aging process that may result in reduced fertility and cognitive functionality. Solutions known in the art typically seek to provide the substrates of musculoskeletal, gonadal or cerebral architecture with supra-normal doses, which assume that saturating metabolic pathways with excess material will push the equation towards repair and regeneration and away from the process of decline and deterioration. It is therefore an objective of the instant disclosure to provide a framework of principles that underly the compositional design of health supplements focused upon the area of connective tissue health (i.e. muscle, joint, and ligament wellness) and reproductive vitality.

Herbal preparations have been shown to have a beneficial effect on activating the toxic-radical neutralization pathways of the body that has statistical significance. These herbal molecules belong to a heterogenous family of molecules known as “polyphenols” and share a common chemical structural architecture characterized by the presence of large multiples of phenol structural units. For example, the health promoting benefits of pomegranate juice, olives, tomatoes, grapes, ginger, turmeric, cinnamon, black pepper, and red chili pepper are now recognized to be in large part due to their polyphenol content.

The herbal extracts of turmeric (Curcuma longa), cinnamon (Cinnamomum verum, Cinnamomum cassia and other members of the same species), black pepper (Piper nigrum), Boswellia serrata (frankincense) and devil's claw root (Harpagophytum procumbens and related species) and red chili pepper (Capsicum chinense and related species) contain polyphenols (curcumin, cinnamaldehyde, piperine, boswellic acids, harpagosides, capsaicin respectively) are known to activate genetic and metabolic pathways. Such genetic and metabolic pathways in turn regulate the expression of the toxic free radical scavenging system of superoxide dismutase, catalase, glutathione reductase and DNA repair mechanisms at a cellular level. Such pathways are also known to participate in the inhibition of the expression of the pro-inflammatory agent, tumor necrosis factor-alpha (“TNF-alpha”), and cyclo-oxygenase-2 (“COX-2”) enzymatic pathway of the inflammatory cascade. Even more, such pathways are known to participate in the activation of nuclear factor erythroid 2-related factor 2 (“NRF2”) and nuclear factor kappa-light-chain-enhancer of activated B cells (“NF-κB”) nuclear transcription factors, which are implicated in the genesis and maintenance of the inflammatory and immune response to various triggers.

Similarly, there has been a growing amount of evidence of the benefits of freeze dried extracts of marine biologicals, especially the extracts made from the meat of green-lipped mussels (Perna canaliculus), sea cucumbers (Holothuriidae), and the recognition of the value of both collagen and high molecular weight sodium hyaluronate in the maintenance the structural integrity of muscle, ligament and joint architecture. Similarly extracts of the photosynthetic regions of plants are known to be rich in cerebrosides, which are also found cerebral and neuronal tissue. For example, extracts of green-lipped mussel and sea cucumbers are rich in glucosamine, chondroitin sulfate, glycose aminoglycans (“GAG”) and collagen.

High molecular weight sodium hyaluronate, which is found in synovial fluid where it functions as joint lubricant, is a major component of avian egg membrane and may be synthetically prepared. Oral consumption of the aforementioned compounds individually is known to have beneficial effects on muscle, ligament and joint health with documented evidence of increased mobility and decreased arthritic pain. Lastly, improvements in gut health and gut immunity are believed to have a positive impact on digestion, absorption as well as overall well-being and vitality.

Embodiments disclosed herein seek to provide a novel framework for the design of health (e.g., nutritional and therapeutic) supplements that utilizes the principle of synergy is utilized to produce benefits between classes of biologicals that synergistically use diverse mechanisms to promote reproductive health as well as muscle, joint and ligament therapy and wellness benefits.

Embodiments disclosed herein further seek to disclose a design principle herein referred to as “Absorb-Repair-Metabolite INGestion” or “ARMING” principle. Design solutions known in the art typically utilize supra-normal doses of a particular biologic substrate for a particular biologic function or physiologic process with the expectation that saturating the metabolic pathways of these processes with excess substrate will push the equation towards repair and regeneration and, concomitantly, away from the process of decline and deterioration. This approach fails to address inflammation, which is a fundamental feature of injury and aging at the tissue level. For example, sore muscles, joints, and/or ligaments (i.e. connective tissue) reflect tissue level inflammation.

Here, simply providing connective tissue with high doses of collagen, hyaluronate, glucosamine, chondroitin sulfate, protein and other building blocks of connective tissue typically will not result in a restoration of functional integrity until inflammation itself is reduced. For example, an attempt to build a house while there is an active fire that is burning the house is not an efficient solution. The most efficient way forward entails first extinguishing the fire prior to initiating the building phase. In other words, simply providing the necessary lumber and other raw materials is not adequate and further requires that these materials are actually delivered to the construction site. In the same vein, providing high doses of various tissue substrates is not enough to initiate repair and requires optimization of oral absorption and gut health. The aforementioned principles form the basis of the ARMING design principle.

The ARMING design principle groups components into three (3) broad categories: components that facilitate oral absorption and gut health; components that neutralize or actively block tissue inflammation or damage; and components that provide optimal amounts of building block nutrients or energy substrates that can be directly used to provide target tissue metabolic, functional and structural integrity. The ARMING design principle can also be used to design health supplements that promote fertility and reproductive health. In addition, the ARMING design principle can be used to design supplements that target cerebral and neuronal function as well as cognitive decline. Here, synergistic quantities of cerebrosides and other substrates of neuronal architecture and function are supplied in conjunction with components that facilitate oral absorption and promote gut health as well as neutralize or actively block tissue inflammation and/or damage.

There presently exists a need for connective tissue (e.g., muscles, ligaments and joints) health supplements that accomplishes the above. Similarly, a need exists for health supplements that promote general reproductive health. As used herein, “composition” refers to health supplement compositions prepared according to the ARMING design principle or ARB system disclosed herein. Preferred embodiments seek to provide compositions for orally administered health supplements. However, in accordance with other embodiments, the compositions are delivered to target musculoskeletal areas using, for example, topical adhesive patches, occlusive or non-occlusive dressings, electromotive force driven transdermal delivery systems, nanoparticle delivery systems, and/or liposomal delivery systems.

In preferred embodiments, the composition is prepared from several herbs and animal biologics that exhibit muscle, joint and ligament or reproductive fertility wellness, analgesic, and antioxidant properties. In some embodiments, the composition is configured to promote muscle relaxation, blood flow, and joint mobility. In other embodiments, the composition is configured to promote increases in sperm count and ovulation.

Preferred embodiments disclosed herein seek to provide a process that combines the following three (3) classes of agents: agents endogenous to a specific system's function; agents that promote gut health and absorption; and agents that block micro-inflammation and toxic radical injury. This system is herein referred to as “ARB” (Absorption-Repair-Build) and includes formulations of the composition that (i) promote muscle, ligament, and joint health, (ii) agents that promote male reproductive health, and (iii) agents that promote female reproductive health, in accordance with preferred embodiments. Formulations that promote connective tissue health include the following: muscle, joint, and ligament agents, anti-inflammatory and antioxidant agents; and gut health probiotic agents and minerals. Here, the muscle, joint, and ligament agents preferably include green-lipped mussel powder (Perna canaliculus), sea cucumber powder (Holothuroidea); sodium hyaluronate (high molecular weight); MSM (methylsulfonylmethane); and creatine monohydrate.

The anti-inflammatory and antioxidant agents preferably consist of Boswellia serrata extract powder or Boswellic acid powder; devil's claw root extract powder (Harpagophytum procumbens); turmeric or curcumin powder (Curcuma longa); Piper nigrum or Piperine powder; calcined curcumin powder; cinnamon extract powder (Cinnamomum verum or Cinnamomum cassia); and ascorbic acid powder. The gut health, pro-absorptive agents and minerals preferably consists of probiotic powder (Lactobacillus species), diatomaceous earth powder, calcined dolomite powder, and molasses powder.

Although alternative formulations may be utilized, Table 1, includes a preferred formulation for the Muscle, Joint, and Ligament Supplement.

TABLE 1

| | |
|---|---|
| green-lipped mussel powder | 1 mg-5 mg/ |
| (Perna canaliculus) | kg body weight |
| sea cucumber powder | 0.5 mg-4 mg/ |
| (holothuroidea) | kg body weight |
| sodium hyaluronate (high molecular weight) | 0.5 mg-4 mg/ |
| | kg body weight |
| MSM (methylsulfonylmethane) | 1 mg-5 mg/ |
| | kg body weight |
| creatine monohydrate | 0.5 mg-4 mg/ |
| | kg body weight |
| Boswellia serrata extract powder or | 1 mg-5 mg/ |
| Boswellic acid | kg body weight |
| devil's claw extract powder | 1 mg-6 mg/ |
| (Harpagophytum procumbens) | kg body weight |
| turmeric/curcumin powder | 0.1 mg-1 mg/ |
| (Curcuma longa) | kg body weight |
| Piper nigrum/piperine powder | 0.05 mg-0.2 mg/ |
| | kg body weight |
| calcined curcumin powder | 0.01 mg-0.1 mg/ |
| | kg body weight |
| cinnamon extract powder | 0.1 mg-1 mg/ |
| (Cinnamomum verum or | kg body weight |
| Cinnamomum cassia) | |
| ascorbic acid powder | 0.5 mg-3 mg/ |
| | kg body weight |
| Lactobacillus probiotic powder | 75,000-150,000 Colony |
| | Forming Units (CFU)/kg |
| | body weight |
| diatomaceous earth powder | 0.5 mg-4 mg/ |
| | kg body weight |
| calcined dolomite powder | 0.25 mg-2 mg/ |
| | kg body weight |
| molasses powder | 1000 mg-4000 mg/ |
| | kg body weight |

In other embodiments, the composition can be formulated as health supplement that addresses male or female reproductive fertility. Here, the male-oriented reproductive fertility formulation preferably includes male gonadogenetic substrate sources, anti-inflammatory and antioxidant agents, and pro-absorptive agents and mineral sources. In preferred embodiments, the male gonadogenetic substrate sources consist of green-lipped mussel powder; maca root extract powder (Lepidium meynii); and Cornus fruit (Shan Zhu Yu) extract powder. The anti-inflammatory and antioxidant agents preferably consist of turmeric/curcumin powder (Curcuma Longa); calcinated curcumin; Piper nigrum/piperine powder; Cinnamomum verum/Cinnamomum cassia powder extract; ginger root extract powder (Zingiber officinale); and ascorbic acid. Similarly, the pro-absorptive agents and mineral source preferably consists of diatomaceous earth; and molasses powder.

Although alternative formulations may be utilized, Table 2, includes a preferred formulation for the male-oriented reproductive fertility supplement.

TABLE 2

| | |
|---|---|
| green-lipped mussel powder | 1 mg-5 mg/ |
| (GLM) | kg body weight |
| maca root powder extract | 0.5 mg-3 mg/ |
| (Lepidium meynii) | kg body weight |
| Cornus fruit extract powder | 0.5 mg-5 mg/ |
| (shan zhu yu) | kg body weight |
| turmeric/curcumin powder | 0.1 mg-1 mg/ |
| (Curcuma Longa) | kg body weight |
| calcinated curcumin | 0.01 mg-0.1 mg/ |
| | kg body weight |
| Piper nigrum/Piperine | 0.05 mg-0.2 mg/ |
| | kg body weight |
| Cinnamomum verum/ | 0.1 mg-1 mg/ |
| Cinnamomum cassia | kg body weight |
| extract powder | |
| ginger root extract powder | 0.5 mg-3 mg/ |
| (Zingiber officinale) | kg body weight |
| ascorbic acid extract powder | 0.5 mg-3 mg/ |
| | kg body weight |
| diatomaceous earth extract powder | 0.5 mg-4 mg/ |
| | kg body weight |
| molasses powder | 1000 mg-4000 mg/ |
| | kg body weight |

Similarly, the female-oriented reproductive fertility formulations preferably include female gonadogenetic substrate sources, anti-inflammatory and antioxidant agents, as well as pro-absorptive agents and mineral sources. Here, the female gonadogenetic substrate sources preferably consist of green-lipped mussel powder; Dioscorea polystachya root extract powder (Chinese yam extract powder, shanyao), shatavari root extract powder (Asparagus racemosus); and black cohosh extract powder (Actaea racemosa). The anti-inflammatory and antioxidant agents preferably consist of ginger root extract powder (Zingiber officinale); broccoli extract powder; turmeric/curcumin powder (Curcuma Longa); calcinated curcumin powder; Piper nigrum/piperine powder extract; Cinnamomum verum/Cinnamomum cassia extract powder; and ascorbic acid powder. The pro-absorptive agents and mineral sources consist of diatomaceous earth powder and ascorbic acid.

Although alternative formulations may be utilized, Table 3, includes a preferred formulation for the female-oriented reproductive fertility supplement.

TABLE 3

| | |
|---|---|
| green-lipped mussel powder | 1 mg-5 mg/ |
| (Perna canaliculus) | kg body weight |
| Dioscorea root | 0.5 mg-3 mg/ |
| (wild yam extract, Shan Yao) | kg body weight |
| shatavari root extract powder | 0.5 mg-5 mg/ |
| (Asparagus racemosus) | kg body weight |
| black cohosh root extract powder | 0.5 mg-5 mg/ |
| (Actea racemose) | kg body weight |
| ginger root extract powder | 0.5 mg-5 mg/ |
| (Zingiber officinale) | kg body weight |
| broccoli extract powder | 1 mg-5 mg/ |
| | kg body weight |
| turmeric/curcumin powder | 0.1 mg-1 mg/ |
| (Curcuma Longa) | kg body weight |
| calcinated curcumin | 0.01 mg-0.1 mg/ |
| | kg body weight |
| Piper nigrum/ | 0.05 mg-0.2 mg/ |
| Piperine extract powder | kg body weight |
| Cinnamomum verum/ | 0.1 mg-1 mg/ |
| Cinnamomum cassia extract powder | kg body weight |
| ascorbic acid | 0.1 mg-1 mg/ |
| | kg body weight |
| diatomaceous earth | 0.1 mg-0.1 mg/ |
| | kg body weight |
| molasses powder | 0.05 mg-0.2 mg/ |
| | kg body weight |

Although the invention has been explained in relation to its preferred embodiment, it is to be understood that many other possible modifications and variations can be made without departing from the spirit and scope of the invention.

1. A health supplement for mammals, the supplement consisting of:
a metabolite ingestion component;
an anti-inflammatory component;
an oral absorption component;
wherein
the metabolite ingestion component comprises green-lipped mussel powder in an amount between 1-5 mg/kg of body weight;
the anti-inflammatory component comprises:
cinnamon powder in an amount between 0.1-1 mg/kg of body weight;
ascorbic acid powder,
turmeric powder present in an amount between 0.1-1 mg/kg of body weight;
Piper nigrum powder in an amount between 0.05-0.2 mg/kg of body weight; and
the oral absorption component comprises diatomaceous earth and molasses powder.

2. The supplement of claim 1, wherein the metabolite ingestion component comprises:
sea cucumber powder;
sodium hyaluronate powder;
methylsylfonylmethane powder; and
creatine monohydrate powder.

3. The supplement of claim 2, wherein the anti-inflammatory component comprises:
Boswellia serrata powder;
Harpagophytum procumbens powder; and
calcined curcumin powder.

4. The supplement of claim 3, wherein the oral absorption component comprises:
a Lactobacillus probiotic powder; and
calcined dolomite powder.

5. The supplement of claim 4, wherein
the sea cucumber powder is in an amount between 0.5-4 mg/kg of body weight;
the sodium hyaluronate powder is in an amount between 0.5-4 mg/kg of body weight;
the methylsylfonylmethane powder is in an amount between 1-5 mg/kg of body weight; and
the creatine monohydrate powder is in an amount between 0.5-4 mg/kg of body weight.

6. The supplement of claim 5, wherein
the Boswellia serrata powder is in an amount between 1-5 mg/kg of body weight
the Harpagophytum procumbens powder is in an amount between 1-6 mg/kg of body weight;
the calcined curcumin powder is in an amount between 0.01-0.1 mg/kg of body weight; and
the ascorbic acid powder is in an amount between 0.5-3 mg/kg of body weight.

7. The supplement of claim 6, wherein
the Lactobacillus probiotic powder is in an amount of 75,000-150,000 CFU;
the calcined dolomite powder is in an amount between 0.25-2 mg/kg of body weight;
the diatomaceous earth powder is in an amount between 0.5-4 mg/kg of body weight; and
the molasses powder is in an amount between 1,000-4,000 mg/kg of body weight.

8. The supplement of claim 1, wherein the metabolite ingestion component comprises:
maca root extract powder; and
Cornus fruit extract powder.

9. The supplement of claim 8, wherein the anti-inflammatory component comprises:
calcined curcumin; and
ginger root extract powder.

10. The supplement of claim 9, wherein
the maca root extract powder is in an amount between 0.5-3 mg/kg of body weight; and
the Cornus fruit extract powder is in an amount between 0.5-5 mg/kg of body weight.

11. The supplement of claim 10, wherein
the turmeric powder is in an amount between 0.1-1 mg/kg of body weight;
the calcined curcumin is in an amount between 0.01-0.1 mg/kg of body weight;
the Piper nigrum powder is in an amount between 0.05-0.2 mg/kg of body weight;
the ginger root extract powder is in an amount between 0.5-3 mg/kg of body weight; and
the ascorbic acid is preset at 0.5-3 mg/kg of body weight.

12. The supplement of claim 11, wherein
the diatomaceous earth is in an amount between 0.5-4 mg/kg of body weight; and
the molasses powder is in an amount between 1,000-4,000 mg/kg of body weight.

13. The supplement of claim 1, wherein the metabolite ingestion component comprises:
wild yam extract powder;
shatavari root extract powder; and
black cohosh extract powder.

14. The supplement of claim 13, wherein the anti-inflammatory component comprises:
ginger root powder;
broccoli extract powder; and
calcined curcumin.

15. The supplement of claim 14, wherein
the wild yam extract powder is preset at 0.5-3 mg/kg of body weight;
the shatavari root extract powder is preset at 0.5-5 mg/kg of body weight; and
the black cohosh extract powder is preset at 0.5-5 mg/kg of body weight.

16. The supplement of claim 15, wherein
the ginger root powder extract is preset at 0.5-3 mg/kg of body weight; and
the broccoli extract powder is preset at 1-5 mg/kg of body weight;
the turmeric powder is in an amount between 0.1-1 mg/kg of body weight;
the calcined curcumin powder is preset at 0.01-0.1 mg/kg of body weight;
the Piper nigrum powder is in an amount between 0.05-0.2 mg/kg of body weight; and
the ascorbic acid is preset at 0.1-1 mg/kg of body weight.

17. The supplement of claim 16, wherein
the diatomaceous earth is in an amount between 0.01-0.1 mg/kg of body weight; and
the molasses powder is in an amount between 0.05-0.2 mg/kg of body weight.
